(19) United States
(12) Patent Application Publication
(10) Pub. No.: US 2021/0341453 A1
(43) Pub. Date: Nov. 4, 2021

(54) Method and System for Estimating Groundwater Recharge Based on Pixel Scale

(71) Applicant: Institute of Geochemistry, Chinese Academy of Sciences, Guiyang (CN)

(72) Inventors: Xiaoyong BAI, Guiyang (CN); Shijie WANG, Guiyang (CN); Luhua WU, Guiyang (CN); Fei CHEN, Guiyang (CN); Miao ZHOU, Guiyang (CN); Yichao TIAN, Guiyang (CN); Guangjie LUO, Guiyang (CN); Qin LI, Guiyang (CN); Jinfeng WANG, Guiyang (CN); Yuanhuan XIE, Guiyang (CN); Yujie Yang, Guiyang (CN); Chaojun LI, Guiyang (CN); Yuanhong DENG, Guiyang (CN); Zeyin HU, Guiyang (CN); Shiqi TIAN, Guiyang (CN); Qian LU, Guiyang (CN); Chen RAN, Guiyang (CN); Min LIU, Guiyang (CN)

(73) Assignee: Institute of Geochemistry, Chinese Academy of Sciences, Guiyang (CN)

(21) Appl. No.: 16/863,877

(22) Filed: Apr. 30, 2020

(51) Int. Cl. G01N 33/24; G06T 7/62

(52) U.S. Cl. CPC G01N 33/246; G06T 7/62

(57) ABSTRACT

Methods and systems for estimating a groundwater recharge based on a pixel scale are disclosed. In some embodiments, a method includes the following steps: (1) obtaining an original remote sensing dataset of a climate element in a study area and a pixel area of the study area; (2) calculating a total water resource yield in the study area by a water balance equation according to the original remote sensing dataset of the climate element and the pixel area of the study area; and (3) estimating the groundwater recharge in the study area according to the total water resource yield and the monthly runoff in the study area. The original remote sensing dataset of the climate element includes monthly precipitation per unit pixel area, monthly actual evapotranspiration per unit pixel area, monthly snowmelt per unit pixel area, monthly soil moisture change per unit pixel area, and monthly runoff.

### FIELD OF THE DISCLOSURE

The disclosure relates generally to ground water recharge estimation. More specifically, the disclosure relates to methods and systems for estimating a groundwater recharge based on a pixel scale.

### BACKGROUND

Groundwater is a water resource as equally important as surface water; and it has a large storage volume and a high exploitation value. However, due to climate change and human activities, groundwater recharge changes dynamically and tends to decline year by year. At present, groundwater storage is mainly estimated based on the real-time monitoring data of groundwater outlets or springs. Because groundwater is deeply buried, its monitoring is difficult and time-consuming, the data from the monitoring points is insufficient, and the monitoring area is small. As a result, such estimation method has great uncertainty on the space-time scale and is difficult to characterize the true characteristics of groundwater changes. Since the groundwater resource has fluidity and recharge performance on a large scale, groundwater resource evaluation often requires crossing provinces and countries. In addition, the storage of groundwater recharge has extremely significant spatial and temporal heterogeneity. Therefore, it is urgent to implement quick, efficient and accurate monitoring to acquire data for analysis. However, the current technologies and methods are difficult to achieve space monitoring tasks, and this brings great difficulties to real-time evaluation of groundwater resource. As a result, a new method for estimating a groundwater recharge quickly, efficiently, and accurately is urgently needed.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provides a method for estimating a groundwater recharge based on a pixel scale. The method includes the following steps.

(1) Obtaining an original remote sensing dataset of a climate element in a study area and a pixel area of the study area. The original remote sensing dataset of the climate element includes monthly precipitation per unit pixel area, monthly actual evapotranspiration per unit pixel area, monthly snowmelt per unit pixel area, monthly soil moisture change per unit pixel area, and monthly runoff.

(2) Calculating a total water resource yield in the study area by a water balance equation according to the original remote sensing dataset of the climate element and the pixel area of the study area.

(3) Estimating the groundwater recharge in the study area according to the total water resource yield and the monthly runoff in the study area.

Optionally, the method further includes step (1a) of preprocessing data in the original remote sensing dataset of the climate element in step (1) to obtain a processed dataset of the climate element in the study area before step (2). Step (1a) includes at least one operation step selected from the group consisting of format conversion, image correction, cropping, registration, quality inspection, and projection conversion.

Optionally, the water balance equation is as follows.

S(Q_{SN}+P)=S(ET+ΔS)+R+G

In the above equation, S is the pixel area and is measured in m², Q_{SN} is the monthly snowmelt per unit pixel area and is measured in mm, P is the monthly precipitation per unit pixel area and is measured in mm, ET is the monthly actual evapotranspiration per unit pixel area and is measured in mm, ΔS is the monthly soil moisture change per unit pixel area and is measured in mm, R is the monthly runoff and is measured in m³, and G is the groundwater recharge and is measured in m³.

Optionally, step (2) includes calculating the total water resource yield in the study area according to the following equation.

W=R+G=S(Q_{SN}+P−ET−ΔS)

In the above equation, W is the total water resource yield in the study area and is measured in m³, R is the monthly runoff in the processed dataset of the climate element and is measured in m³, G is the groundwater recharge and is measured in m³, S is the pixel area and is measured in m², Q_{SN} is the monthly snowmelt per unit pixel area in the processed dataset of the climate element and is measured in mm, P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

Optionally, step (3) includes estimating the groundwater recharge in the study area according to the following equation.

G=W−R=S(Q_{SN}+P−ET−ΔS)−R

In the above equation, G is the groundwater recharge in the study area and is measured in m³, W is the total water resource yield in the study area and is measured in m³, R is the monthly runoff in the processed dataset of the climate element and is measured in m³, S is the pixel area and is measured in m², Q_{SN} is the monthly snowmelt per unit pixel area in the processed dataset of the climate element and is measured in mm; P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

In other embodiments, the disclosure provides a system for estimating a groundwater recharge based on a pixel scale. The system includes an information obtaining module, a study area total water resource yield calculation module, and a groundwater recharge estimation module.

The information obtaining module is configured to obtain an original remote sensing dataset of a climate element in a study area and a pixel area of the study area. The original remote sensing dataset of the climate element includes monthly precipitation per unit pixel area, monthly actual evapotranspiration per unit pixel area, monthly snowmelt per unit pixel area, monthly soil moisture change per unit pixel area, and monthly runoff.

The study area total water resource yield calculation module is configured to calculate a total water resource yield in the study area by a water balance equation according to the original remote sensing dataset of the climate element and the pixel area of the study area.

The groundwater recharge estimation module is configured to estimate the groundwater recharge in the study area according to the total water resource yield and the monthly runoff in the study area.

Optionally, the system further includes a preprocessing module configured to preprocess data in the original remote sensing dataset of the climate element to obtain a processed dataset of the climate element in the study area. The preprocessing includes at least one operation step selected from the group consisting of format conversion, image correction, cropping, registration, quality inspection and projection conversion.

Optionally, the study area total water resource yield calculation module includes a study area total water resource yield calculation unit configured to calculate the total water resource yield in the study area according to the following equation.

W=R+G=S(Q_{SN}+P−ET−ΔS)

In the above equation, W is the total water resource yield in the study area and is measured in m³, R is the monthly runoff in the processed dataset of the climate element and is measured in m³, G is the groundwater recharge in the study area and is measured in m³, S is the pixel area and is measured in m², Q_{SN} is the monthly snowmelt per unit pixel area in the processed dataset of the climate element and is measured in mm; P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

Optionally, the groundwater recharge estimation module includes a groundwater recharge estimation unit configured to estimate the groundwater recharge in the study area according to the following equation.

G=W−R=S(Q_{SN}+P−ET−ΔS)−R

In the above equation, G is the groundwater recharge in the study area and is measured in m³, W is the total water resource yield in the study area and is measured in m³, R is the monthly runoff in the processed dataset of the climate element and is measured in m³, S is the pixel area and is measured in m², Q_{SN} is the monthly snowmelt per unit pixel area in the processed dataset of the climate element and is measured in mm; P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

### BRIEF DESCRIPTION OF THE DRAWINGS

Illustrative embodiments of the present disclosure are described in detail below with reference to the figures.

FIG. 1 is a flowchart illustrating a method for estimating a groundwater recharge based on a pixel scale according to an embodiment of the disclosure.

FIG. 2 is a structural diagram illustrating a system for estimating a groundwater recharge based on a pixel scale according to an embodiment of the disclosure.

### DETAILED DESCRIPTION

The following describes some non-limiting embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

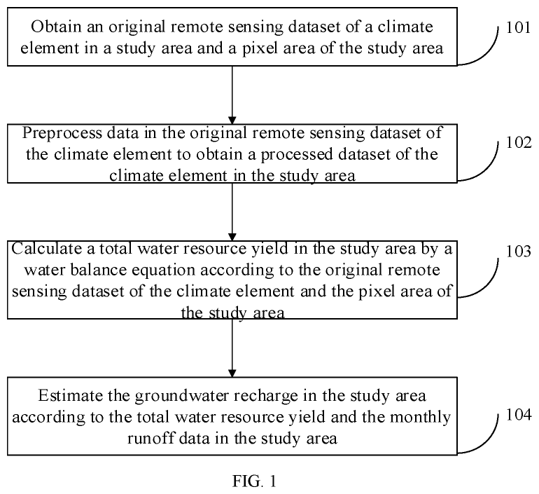

FIG. 1 is a flowchart illustrating a method for estimating a groundwater recharge based on a pixel scale according to an embodiment of the disclosure. As shown in FIG. 1, the disclosure may provide a method for estimating a groundwater recharge based on a pixel scale including the following steps 101-104.

Step 101. Obtaining an original remote sensing dataset of a climate element in a study area and a pixel area of the study area. The original remote sensing dataset of the climate element may include global land water storage change data from Gravity Recovery and Climate Experiment (GRACE), monthly precipitation per unit pixel area, monthly actual evapotranspiration per unit pixel area, monthly snowmelt per unit pixel area, monthly soil moisture change per unit pixel area, monthly runoff, et cetera.

The original remote sensing dataset of the climate element may include the following monthly data per unit pixel area: precipitation, actual evapotranspiration, soil water content at thicknesses of 0-10 cm, 10-40 cm, 40-100 cm, and 100-200 cm, and snowmelt, which may be merged into annual data. These data may be derived from a dataset of the Famine Early Warning Systems Network Land Data Assimilation System (FLDAS) (FLDAS Noah Land Surface Model L4 Global Monthly 0.1×0.1 degree (MERRA-2 and CHIRPS) V001 (FLDAS_NOAH01_C_GL_M) at GES DISC (https://ldas.gsfc.nasa.gov/FLDAS/)) of National Aeronautics and Space Administration (NASA) (https://www.nasa.gov/). The FLDAS dataset has a spatial resolution of 0.1°×0.1°. These data may also be derived from a dataset of the Global Land Data Assimilation System (GLDAS). The original remote sensing dataset of the climate element may have a time resolution of monthly and may have a global spatial coverage (60S, 180W, 90N and 180E).

In addition, global soil depth may be used to calculate the monthly soil thickness. The global soil depth may be derived from https://daac.ornl.gov/, with a spatial resolution of 0.1°×0.1°, and may also be derived from https://www.isric.org/explore/soilgrids. Soil depth with different spatial resolutions may be selected according to a research scale of 250 m×250 m, 1 km×1 km, 5 km×5 km, and 10 km×10 km. The latest administrative division vector data in 2015 may be derived from the Resource and Environment Data Cloud Platform of the Chinese Academy of Sciences (http://www.resdc.cn/) and the National Bureau of Surveying, Mapping, and Geographic Information (http://www.sbsm.gov.cn/article/zxbs/dtfw/).

Global land snowmelt and surface runoff may be derived from the GLDAS (the Goddard Earth Sciences Data and Information Services Center (GES DISC) (GLDAS Noah Land Surface Model L4 Monthly 0.25×0.25 degree) https://mirador.gsfc.nasa.gov/)).

Step 102. Preprocessing data in the original remote sensing dataset of the climate element to obtain a processed dataset of the climate element in the study area. The preprocessing may include at least one operation step selected from the group consisting of format conversion, image correction, cropping, registration, quality inspection, and projection conversion.

The disclosure utilizes a data assimilation method to convert a grid cell size of all raster data in the original remote sensing dataset of the climate element to the same scale. The projection method may be Albers Equal-area Conic Projection (Krasovsky-1940-Albers), which is a projected coordinate system.

The above-mentioned global scale raster data may be processed by format conversion, image correction, cropping, registration, quality inspection, and projection conversion to finally obtain a processed dataset of the climate element in the study area.

Step 103. Calculating a total water resource yield in the study area by a water balance equation according to the original remote sensing dataset of the climate element and the pixel area of the study area. The calculation may include following steps. First, deriving a calculation formula for the total water resource yield in the study area based on the water balance equation. Second, inputting the pixel area of the study area and the monthly snowmelt per unit pixel area, the monthly precipitation per unit pixel area, the actual evapotranspiration per unit pixel area, and the monthly soil moisture change per unit pixel area in the processed dataset of the climate element to the calculation formula for the total water resource yield in the study area to determine the total water resource yield of the study area.

The water balance equation (1) may be as follows.

S(Q_{SN}+P)=S(ET+ΔS)+R+G (1)

In the above equation (1), S is the pixel area and is measured in m², Q_{SN} is the monthly snowmelt per unit pixel area and is measured in mm, P is the monthly precipitation per unit pixel area and is measured in mm, ET is the monthly actual evapotranspiration per unit pixel area and is measured in mm, ΔS is the monthly soil moisture change per unit pixel area and is measured in mm, R is the monthly runoff and is measured in m³, and G is the groundwater recharge and is measured in m³.

The total water resource yield in the study area may be calculated according to the following equation (2).

W=R+G=S(Q_{SN}+P−ET−ΔS) (2)

In the above equation (2), W is the total water resource yield in the study area and is measured in m³, R is the monthly runoff in the processed dataset of the climate element and is measured in m³, G is the groundwater recharge and is measured in m³, S is the pixel area and is measured in m², Q_{SN} is the monthly snowmelt per unit pixel area in the processed dataset of the climate element and is measured in mm, P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

The snowmelt may be derived from resampling of global (60S, 180W, 90N, 180E) monthly snowmelt per unit pixel area with a spatial resolution of 0.125°×0.125°. The above-mentioned snowmelt may be derived from a dataset of the North American Land Data Assimilation System (NLDAS) (Noah Land Surface Model L4 Monthly 0.125°×0.125°, https://mirador.gsfc.nasa.gov/).

The soil moisture change may be resampled soil water content raster data (soil depth 2 m) derived from a dataset of the FLDAS (FLDAS Noah Land Surface Model L4 Global Monthly 0.1×0.1 degree (MERRA-2 and CHIRPS) V001 (FLDAS_NOAH01_C_GL_M) at GES DISC (https://ldas.gsfc.nasa.gov/FLDAS/)) of the NASA (https://www.nasa.gov/) with a spatial resolution of 0.1°×0.1°.

Step 104. Estimating the groundwater recharge in the study area according to the total water resource yield and the monthly runoff in the study area. The estimation may include the following steps. First, deriving a calculation formula for the groundwater recharge in the study area based on the calculation formula of the total water resource yield in the study area. Second, inputting the total water resource yield in the study area and the monthly runoff in the processed dataset of the climate element to the calculation formula for the groundwater recharge in the study area to determine the groundwater recharge in the study area.

The groundwater recharge in the study area may be calculated according to the following equation (3).

G=W−R=S(Q_{SN}+P−ET−ΔS)−R (3)

In the above equation (3), G is the groundwater recharge in the study area and is measured in m³, W is the total water resource yield in the study area and is measured in m³, R is the monthly runoff in the processed dataset of the climate element and is measured in m³, Q_{SN} is the monthly snowmelt per unit pixel area in the processed dataset of the climate element and is measured in mm, P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

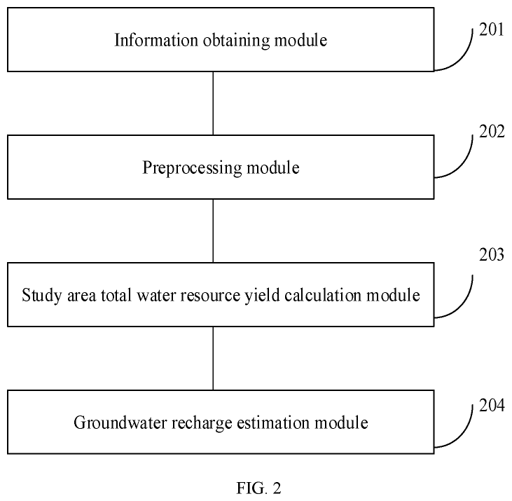

FIG. 2 is a structural diagram illustrating a system for estimating a groundwater recharge based on a pixel scale according to an embodiment of the disclosure. As shown in FIG. 2, the disclosure may provide a system for estimating a groundwater recharge based on a pixel scale including an information obtaining module 201, a preprocessing module 202, a study area total water resource yield calculation module 203, and a groundwater recharge estimation module 204.

The information obtaining module 201 may be configured to obtain an original remote sensing dataset of a climate element in a study area and a pixel area of the study area. The original remote sensing dataset of the climate element may include monthly precipitation per unit pixel area, monthly actual evapotranspiration per unit pixel area, monthly snowmelt per unit pixel area, monthly soil moisture change per unit pixel area, and monthly runoff.

The preprocessing module 202 may be configured to preprocess data in the original remote sensing dataset of the climate element to obtain a processed dataset of the climate element in the study area. The preprocessing may include at least one operation step selected from the group consisting of format conversion, image correction, cropping, registration, quality inspection, and projection conversion. Optionally, the preprocessing may include the operation steps of format conversion, image correction, cropping, registration, quality inspection, and projection conversion in sequence.

The study area total water resource yield calculation module 203 may be configured to calculate a total water resource yield in the study area by a water balance equation according to the original remote sensing dataset of the climate element and the pixel area of the study area.

The groundwater recharge estimation module 204 may be configured to estimate the groundwater recharge in the study area according to the total water resource yield and the monthly runoff in the study area.

The study area total water resource yield calculation module 203 may include a study area total water resource yield calculation unit configured to calculate the total water resource yield in the study area according to following equation (4).

W=R+G=S(Q_{SN}+P−ET−ΔS) (4)

In the above equation (4), W is the total water resource yield in the study area and is measured in m³, R is the monthly runoff in the processed dataset of the climate element and is measured in m³, G is the groundwater recharge and is measured in m³, S is the pixel area and is measured in m², Q_{SN} is the monthly snowmelt per unit pixel area in the processed dataset of the climate element and is measured in mm, P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

The groundwater recharge estimation module 204 may include a groundwater recharge estimation unit configured to estimate the groundwater recharge in the study area according to the following equation (5).

G=W−R=S(Q_{SN}+P−ET−ΔS)−R (5)

In the above equation (5), G is the groundwater recharge in the study area and is measured in m³, W is the total water resource yield in the study area and is measured in m³, R is the monthly runoff in the processed dataset of the climate element and is measured in m³, S is the pixel area and is measured in m², Q_{SN} is the monthly snowmelt per unit pixel area in the processed dataset of the climate element and is measured in mm, P is the monthly precipitation per unit pixel area in the processed dataset of the climate element and is measured in mm, ET is the monthly actual evapotranspiration in the processed dataset of the climate element and is measured in mm, and ΔS is the monthly soil moisture change per unit pixel area in the processed dataset of the climate element and is measured in mm.

Several examples are used herein for illustration of the principles and embodiments of the present invention. The description of the embodiments is used to help illustrate the method and its core principles of the present invention. In addition, a person of ordinary skill in the art can make various modifications in terms of specific embodiments and scope of application in accordance with the teachings of the present invention. In conclusion, the content of this specification shall not be construed as a limitation to the present invention.

Various embodiments of the disclosure may have one or more of the following effects.

In some embodiments, the disclosure may provide a method and a system for estimating a groundwater recharge based on a pixel scale. The disclosure may overcome the technical shortcomings of the existing models and technologies which have difficulties achieving groundwater storage evaluation based on a spatial pixel scale. The disclosure may make up for the technology blank of existing models and technologies.

In other embodiments, the disclosure may provide a method and a system for estimating a groundwater recharge based on a pixel scale. The disclosure may use monthly runoff, monthly precipitation, monthly actual evapotranspiration, and monthly snowmelt to derive a groundwater recharge estimation model based on a water balance equation.

In further embodiments, the disclosure may provide a groundwater recharge estimation model for the monitoring and evaluation of groundwater reserves. The model may be quick, efficient, and applicable to a global large scale, and may help to solve the problem of difficult, time-consuming, and low-accuracy monitoring of groundwater recharge. The model may provide new technical support and theoretical basis for research on ecological restoration and socioeconomic development.

In some embodiments, the disclosure may help to implement the evaluation of groundwater recharge and provide new technical support for socioeconomic development and ecological restoration and construction.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A method for estimating a groundwater recharge based on a pixel scale, comprising the steps of:
(1) obtaining an original remote sensing dataset of a climate element in a study area and a pixel area of the study area, wherein the original remote sensing dataset of the climate element comprises monthly precipitation per unit pixel area, monthly actual evapotranspiration per unit pixel area, monthly snowmelt per unit pixel area, monthly soil moisture change per unit pixel area, and monthly runoff;
(2) calculating a total water resource yield in the study area by a water balance equation according to the original remote sensing dataset of the climate element and the pixel area of the study area; and
(3) estimating the groundwater recharge in the study area according to the total water resource yield and the monthly runoff in the study area.

2. The method of claim 1, wherein:
the method further comprises step (1a) of preprocessing data in the original remote sensing dataset of the climate element in step (1) to obtain a processed dataset of the climate element in the study area before step (2); and
step (1a) includes at least one operation step selected from the group consisting of format conversion, image correction, cropping, registration, quality inspection, and projection conversion.

3. The method of claim 1, wherein the water balance equation is
S(Q_{SN}+P)=S(ET+ΔS)+R+G,
wherein:
S is the pixel area and is measured in m²;
Q_{SN} is the monthly snowmelt per unit pixel area and is measured in mm;
P is the monthly precipitation per unit pixel area and is measured in mm;
ET is the monthly actual evapotranspiration per unit pixel area and is measured in mm;
ΔS is the monthly soil moisture change per unit pixel area and is measured in mm,
R is the monthly runoff and is measured in m³; and
G is the groundwater recharge and is measured in m³.

4. The method of claim 2, wherein step (2) comprises:
calculating the total water resource yield in the study area according to the following equation:
W=R+G=S(Q_{SN}+P−ET−ΔS),
wherein:
W is the total water resource yield in the study area and is measured in m³;
R is the monthly runoff in the processed dataset of the climate element and is measured in m³;
G is the groundwater recharge and is measured in m³;
S is the pixel area and is measured in m²;
Q_{SN} is the monthly snowmelt per unit pixel area in the processed dataset of the climate element and is measured in mm;
P is the monthly precipitation per unit pixel area in the processed dataset of the climate element and is measured in mm;
ET is the monthly actual evapotranspiration in the processed dataset of the climate element and is measured in mm; and
ΔS is the monthly soil moisture change per unit pixel area in the processed dataset of the climate element and is measured in mm.

5. The method of claim 2, wherein step (3) comprises:
estimating the groundwater recharge in the study area according to the following equation:
G=W−R=S(Q_{SN}+P−ET−ΔS)−R,
wherein:
G is the groundwater recharge in the study area and is measured in m³;
W is the total water resource yield in the study area and is measured in m³;
R is the monthly runoff in the processed dataset of the climate element and is measured in m³;
S is the pixel area m²;
Q_{SN} is the monthly snowmelt per unit pixel area in the processed dataset of the climate element and is measured in mm;
P is the monthly precipitation per unit pixel area in the processed dataset of the climate element and is measured in mm;
ET is the monthly actual evapotranspiration in the processed dataset of the climate element and is measured in mm; and
ΔS is the monthly soil moisture change per unit pixel area in the processed dataset of the climate element and is measured in mm.

6. A system for estimating a groundwater recharge based on a pixel scale, comprising:
an information obtaining module configured to obtain an original remote sensing dataset of a climate element in a study area and a pixel area of the study area, wherein the original remote sensing dataset of the climate element comprises monthly precipitation per unit pixel area, monthly actual evapotranspiration per unit pixel area, monthly snowmelt per unit pixel area, monthly soil moisture change per unit pixel area, and monthly runoff;
a study area total water resource yield calculation module configured to calculate a total water resource yield in the study area by a water balance equation according to the original remote sensing dataset of the climate element and the pixel area of the study area; and
a groundwater recharge estimation module configured to estimate the groundwater recharge in the study area according to the total water resource yield and the monthly runoff in the study area.

7. The system of claim 6, further comprising a preprocessing module configured to preprocess data in the original remote sensing dataset of the climate element to obtain a processed dataset of the climate element in the study area, wherein the preprocessing includes at least one operation step selected from the group consisting of format conversion, image correction, cropping, registration, quality inspection and projection conversion.

8. The system of claim 7, wherein the study area total water resource yield calculation module comprises:
a study area total water resource yield calculation unit configured to calculate the total water resource yield in the study area according to the following equation:
W=R+G=S(Q_{SN}+P−ET−ΔS),
wherein:
W is the total water resource yield in the study area and is measured in m³;
R is the monthly runoff in the processed dataset of the climate element and is measured in m³;
G is the groundwater recharge in the study area and is measured in m³;
S is the pixel area and is measured in m²;
Q_{SN} is the monthly snowmelt per unit pixel area in the processed dataset of the climate element and is measured in mm;
P is the monthly precipitation per unit pixel area in the processed dataset of the climate element and is measured in mm;
ET is the monthly actual evapotranspiration in the processed dataset of the climate element and is measured in mm; and
ΔS is the monthly soil moisture change per unit pixel area in the processed dataset of the climate element and is measured in mm.

9. The system of claim 7, wherein the groundwater recharge estimation module comprises:
a groundwater recharge estimation unit configured to estimate the groundwater recharge in the study area according to the following equation:
G=W−R=S(Q_{SN}+P−ET−ΔS)−R;
wherein:
G is the groundwater recharge in the study area and is measured in m³;
W is the total water resource yield in the study area and is measured in m³;
R is the monthly runoff in the processed dataset of the climate element and is measured in m³;
S is the pixel area and is measured in m²;
Q_{SN} is the monthly snowmelt per unit pixel area in the processed dataset of the climate element and is measured in mm;
P is the monthly precipitation per unit pixel area in the processed dataset of the climate element and is measured in mm;
ET is the monthly actual evapotranspiration in the processed dataset of the climate element and is measured in mm; and
ΔS is the monthly soil moisture change per unit pixel area in the processed dataset of the climate element and is measured in mm.
